(19) United States
(12) Patent Application Publication
(10) Pub. No.: US 2026/0354864 A1
(43) Pub. Date: Nov. 12, 2026

(54) DEVICE FOR THE TREATMENT OF SHOULDER DYSTOCIA

(71) Applicant: Everest Medical Solutions, Inc., San Luis Obispo, CA (US)

(72) Inventors: Jenna M. Eissmann, Reno, NV (US); Madeleine M. Mumford, Snoqualmie, WA (US)

(21) Appl. No.: 19/667,017

(22) Filed: May 4, 2026

Related U.S. Application Data
(60) Provisional application No. 63/800,976, filed on May 6, 2025.

(51) Int. Cl. A61B 17/44

(52) U.S. Cl. CPC A61B 17/44

(57) ABSTRACT

A birthing device is used to aid in labor and delivery complications, including shoulder dystocia where an infant's shoulder is impacted in a mother's pubic symphysis. The device includes a pair of panels and a flexible sheet that are usable to compress an infant in a pelvic cavity and remove the infant's shoulder from the mother's pubic symphysis. The device includes a pair of manipulator arms that are coupled to the respective panels so that an operator, such as a medical provider, can control movement of the panels and flexible sheet. The panels define sufficient surface areas that can be effectively used to compress the infant. Such panels may provide an atraumatic shape that facilitates injury prevention of the mother and infant.

### CROSS REFERENCE TO RELATED APPLICATION

The present application claims the benefit of U.S. provisional patent application Ser. No. 63/800,976 filed May 6, 2025, which is hereby incorporated herein by reference in its entirety.

### FIELD

The present disclosure relates to medical devices, and, more particularly, to labor and delivery devices.

### BACKGROUND

Shoulder dystocia is a childbirth complication that can arise during labor and delivery. It occurs when an infant's shoulder is lodged above their mother's pubic or pubic symphysis, preventing the infant from exiting the vaginal canal. A shoulder dystocia emergency can lead to injuries of the mother and infant. The mother could suffer painful injuries or conditions, including severe tearing of the perineum, rectovaginal fistula, uterine rupture, and postpartum hemorrhages. The infant is also at risk. The infant could suffer from brachial plexus palsy, fractures to the collarbone, and fractures to the upper arm bone.

### SUMMARY

A birthing device includes a compression body to facilitate the treatment of shoulder dystocia. The compression body includes a pair of panels that can compress an infant in their mother's pelvic cavity to remove the infant's shoulder from the mother's pubic symphysis, and thereby resolve shoulder dystocia. The panels are sized to provide sufficient surface area for mitigating injury of the infant while undergoing compression. The panels are shaped so that they can be readily inserted in the mother's pelvic cavity, and then readily positioned on the infant for compression. The compression body also includes a flexible sheet that can wrap around the infant to further facilitate compression of the infant and resolution of shoulder dystocia. The device includes a pair of manipulator arms that are coupled to respective panels. The manipulator arms can be moved by an operator, such as a medical provider, to wrap the panels and flexible sheet around the infant. The manipulator arms can then be further moved to cause the panels and flexible sheet to compress the infant for resolving shoulder dystocia.

In one example, a birthing device for treating shoulder dystocia includes a pair of manipulator arms, a pair of panels coupled to the respective arms, and a flexible sheet coupled to the panels. The panels and flexible sheet cooperate with one another to manipulate an infant in a pelvic cavity, such as a human infant in a human's pelvic cavity.

In another example, the flexible sheet is a central flexible sheet. Each of the panels includes a frame and a lateral flexible sheet. The panels' frames are coupled to the respective manipulator arms and the respective lateral flexible sheets. The central and lateral flexible sheets may be unitarily formed, and the central flexible sheet may be coupled to the frames via the respective lateral flexible sheets.

In yet another example, the frames each define a through hole, and the lateral flexible sheets cover the respective through holes. The through holes may have an elongated shape.

In still another example, the frames are planar, and the flexible sheets are rectangular shaped.

In a further example, the panels include respective lateral edge portions. The device has a width extending between the lateral edge portions that is greater than a width of the flexible sheet that extends along the lateral edge portions.

In yet another example, each of the manipulator arms includes a straight shaft. The straight shafts are coupled to the respective panels.

In still another example, each of the manipulator arms includes a first surface and a second surface. The first surfaces each include an arcuate surface, and the second surfaces are planar.

In a further example, the manipulator arms define respective notches that can engage with one another, such as while the device is an undeployed configuration. The device may be in the undeployed configuration while the device is being inserted into the mother's pelvic cavity.

In another example, the flexible sheet defines multiple segments that are adapted to be stacked in an accordion shape, such as while the device is in the undeployed configuration. This may enable rapid positioning of the device into a deployed configuration in which the panels and flexible sheet can compress an infant in a mother's pelvic cavity.

Thus, the birthing device improves treatment for shoulder dystocia. This is accomplished by the device's compression body, including the pair of panels that can compress an infant in a mother's pelvic cavity, while also providing sufficient surface area to mitigate injury of the infant undergoing compression. The compression body also includes the flexible sheet to facilitate compression of the infant. The compression body is shaped such that it can be readily inserted into the mother's pelvic cavity and wrapped around the infant to a position where it can effectively compress the infant for treating shoulder dystocia.

These and other objects, advantages, purposes, and features of this disclosure will become apparent upon review of the following specification in conjunction with the drawings.

### BRIEF DESCRIPTION OF DRAWINGS

FIGS. 1-3 are schematic views of a birthing device being used to treat shoulder dystocia of a human infant;

FIGS. 4-6 are perspective views of a device similar to the device of FIGS. 1-3, the device shown in a fully deployed configuration and including a pair of manipulator arms, a pair of frames, and a flexible sheet;

FIG. 7 is a front elevation view of the device of FIG. 4 in the fully deployed configuration;

FIG. 8 is a back elevation view of the device of FIG. 4 in the fully deployed configuration;

FIG. 9 is a perspective view of one of the manipulator arms and one of the frames of FIG. 4;

FIG. 10 is a back perspective view of the flexible sheet of FIG. 4;

FIG. 11 is a back elevation view of the flexible sheet of FIG. 4;

FIG. 12 is a perspective view of the device of FIG. 4 in a partially deployed configuration;

FIG. 13 is a top plan view of the device of FIG. 4 in the partially deployed configuration;

FIG. 14 is a back elevation view of the device of FIG. 4 in the partially deployed configuration;

FIG. 15 is an enlarged back elevation view of a portion of the device of FIG. 4 in the partially deployed configuration;

FIG. 16 is a perspective view of the device of FIG. 4 in an undeployed configuration;

FIG. 17 is a top plan view of the device of FIG. 4 in the undeployed configuration;

FIG. 18 is a side elevation view of the device of FIG. 4 in the undeployed configuration;

FIG. 19 is a front elevation view of the device of FIG. 4 in the undeployed configuration;

FIGS. 20 and 21 are perspective views of the device of FIG. 4 treating shoulder dystocia of a human infant, the device shown in an undeployed configuration and relative to a mother's pelvis;

FIG. 22 is a perspective view of the device of FIG. 4 treating shoulder dystocia of the infant relative to the mother's pelvis of FIG. 20, the device shown in a fully deployed configuration in which the device's compression body is wrapped around the infant; and

FIG. 23 is a front elevation view of the device of FIG. 4 treating shoulder dystocia of the infant relative to the mother's pelvis of FIG. 20, the device shown in the fully deployed configuration, having compressed and rotated the infant.

### DETAILED DESCRIPTION

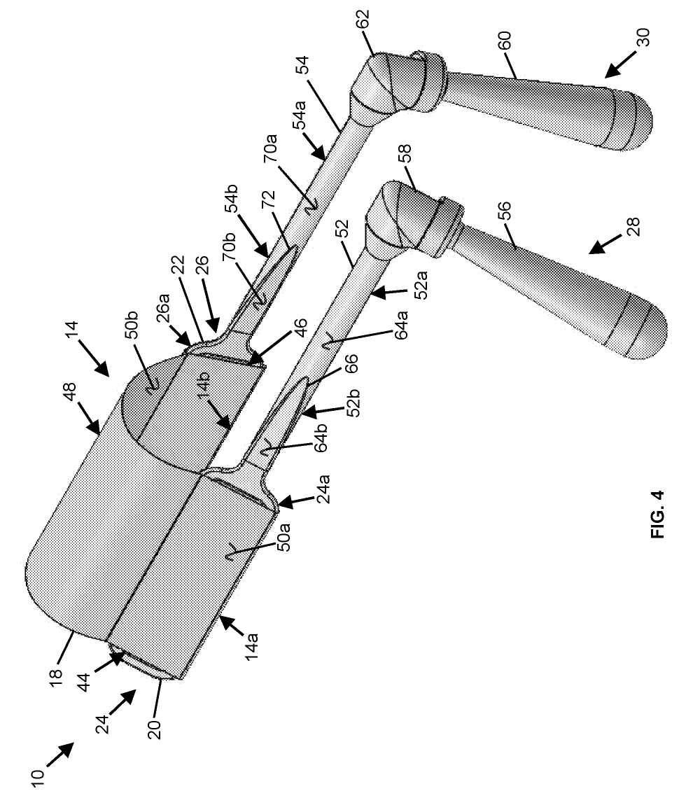

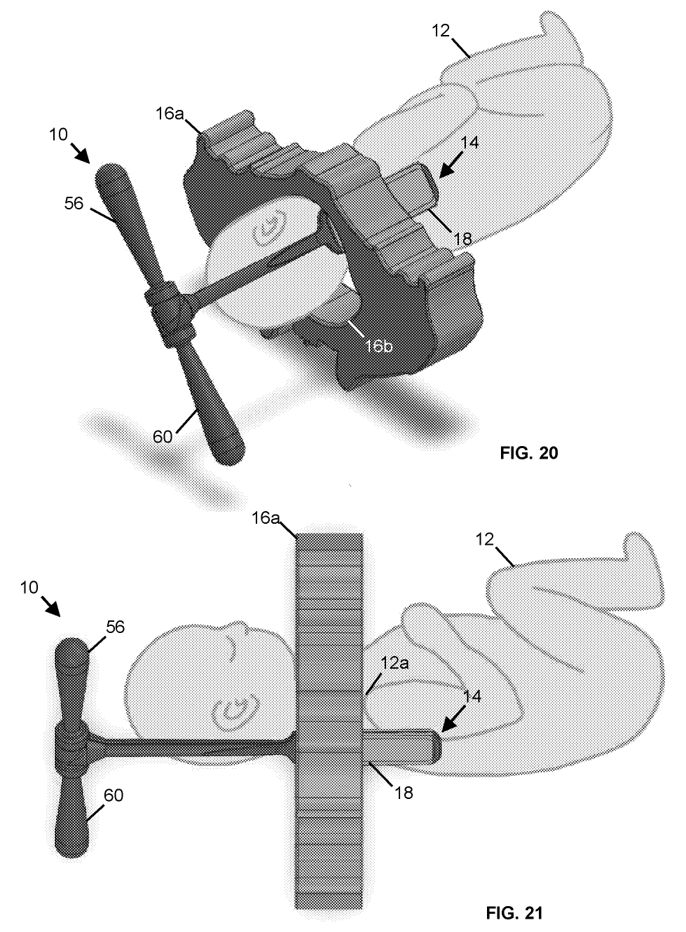

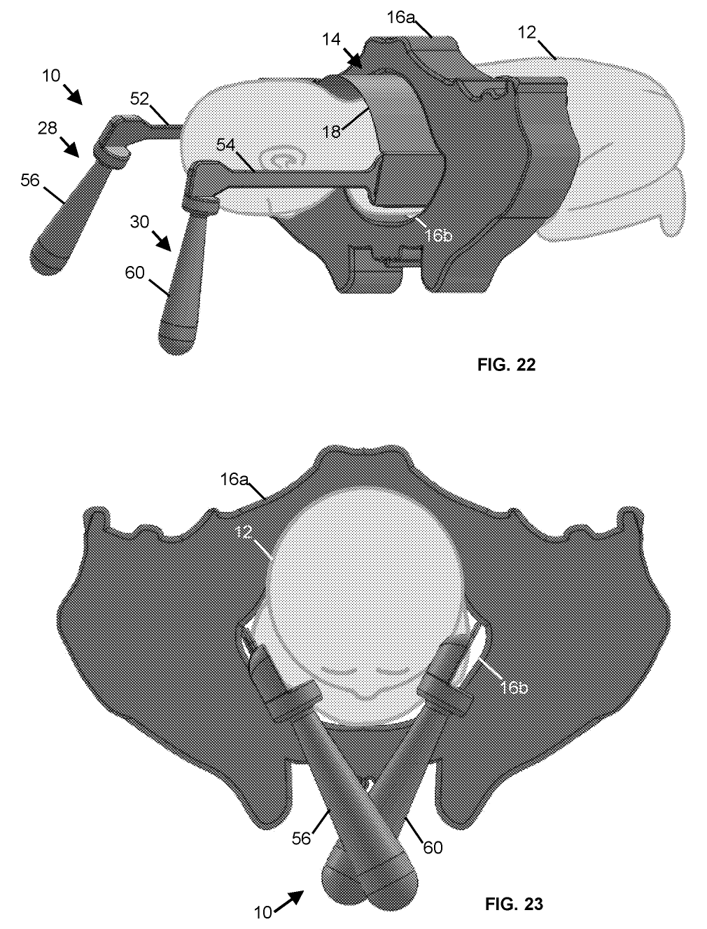

Referring now to the drawings and illustrative examples depicted therein, a birthing device 10 aids in resolving birth complications, such as shoulder dystocia. (FIGS. 4-6). Device 10 is used to manipulate the position of an infant 12 in their mother's pelvic cavity to facilitate safe delivery. (FIGS. 20-23). Device 10 includes a compression body 14 that can swaddle or wrap around infant 12 to compress, rotate, or otherwise manipulate infant 12. (FIGS. 22, 23). For example, in the event shoulder dystocia occurs in which infant's shoulder 12a is engaged with a pubic symphysis of the mother's pelvis 16a, compression body 14 can compress infant 12 to disengage or dislodge shoulder 12a from the pubic symphysis. (FIGS. 20-23). Compression body 14 includes a flexible sheet 18 for compressing infant 12. (FIGS. 4-6). Compression body 14 further includes a left frame 20 and a right frame 22 that support flexible sheet 18 and cooperate with flexible sheet 18 to form a left panel 24 and a right panel 26, respectively. Birthing device 10 includes a left manipulator arm 28 and a right manipulator arm 30 that are directly coupled to respective panels 24, 26 such that arms 28, 30 can be manipulated by an operator, such as a medical provider, to facilitate controlled movement of compression body 14. Panels 24, 26 provide a sufficient surface area that can be effectively used to contact and compress infant 12 to aid in difficult births, such as treating shoulder dystocia, while preventing injuries of mothers and infants.

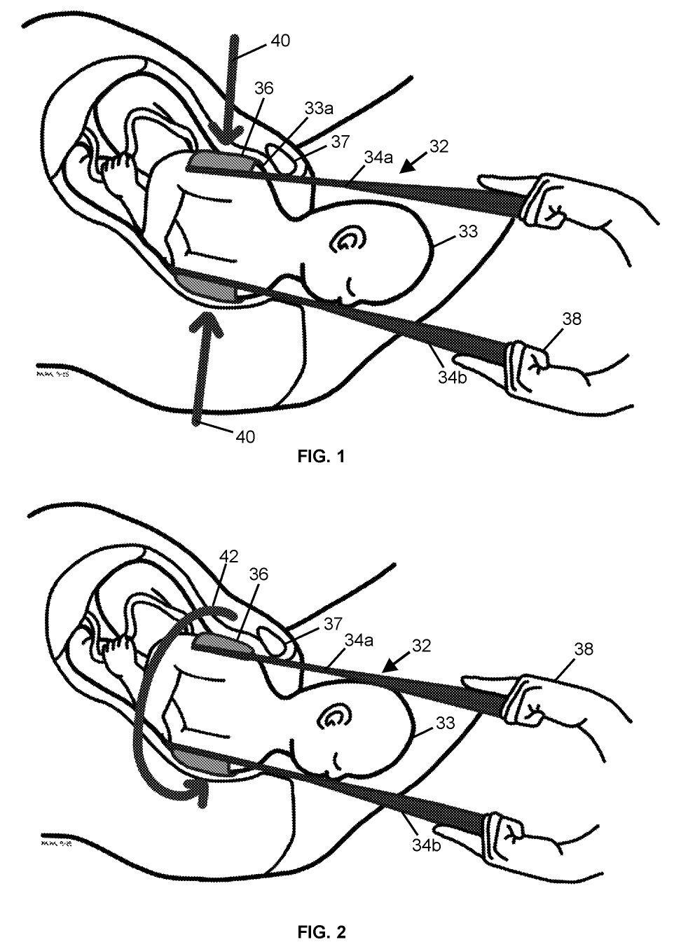

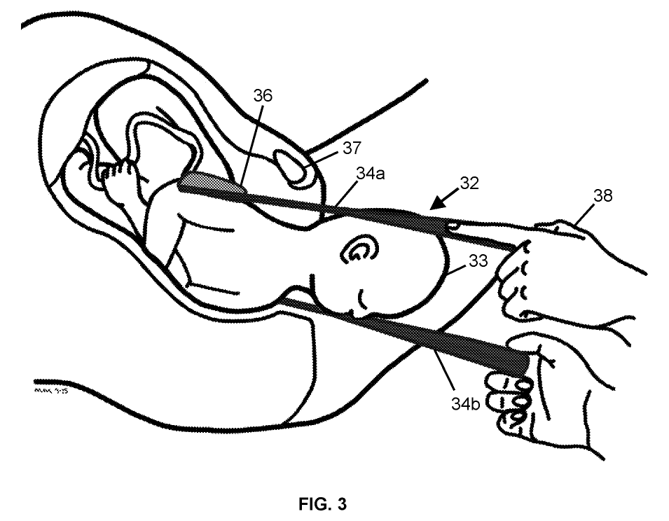

FIGS. 1-3 show the process of using a device 32 similar to device 10 of FIGS. 4-23 for compressing an infant 33 to treat shoulder dystocia. Certain elements of device 32 are omitted and/or appear differently compared to device 10 of FIGS. 4-23 for clarity purposes. Device 32 of FIGS. 1-3 is shown including a left manipulator arm 34a, a right manipulator arm 34b, and a compression body 36 similar to respective manipulator arms 28, 30 and compression body 14 of device 10 of FIGS. 4-23. FIG. 1 shows infant 33 experiencing shoulder dystocia in which a shoulder 33a of infant 33 is impacted or stuck on mother's pubic symphysis 37. To address this, a medical provider 38 moves manipulator arms 34a, 34b to deploy device 32 such that compression body 36 swaddles or wraps a portion of an upper body of infant 33, such as its impacted shoulder girdle, as shown in FIGS. 1-3. Medical provider 38 then applies an adductive or compressive force 40 toward a midline of infant 33 (FIG. 1), to collapse the impacted portion of the upper body of infant 33, as shown in FIG. 2. Medical provider 38 may then apply a rotational force 42 (FIG. 2), while still applying compressive force 40 (FIG. 1), to remove the shoulder of infant 33 from pubic symphysis 37 and resolve shoulder dystocia, as shown in FIG. 3. Infant 33 can then be fully delivered.

Referring again to device 10 of FIGS. 4-23, FIGS. 4-8 show compression body 14 in a fully deployed configuration in which flexible sheet 18 is fully opened and usable to compress an infant, such as in the way described above in connection with FIGS. 1-3. Flexible sheet 18 defines a left sleeve 44 attached to left frame 20, a right sleeve 46 attached to right frame 22, and a central section 48 attached to sleeves 44, 46. (FIG. 4). Frames 20, 22 are planar such that left sleeve 44 and left frame 20 cooperate to form left panel 24, and right sleeve 46 and right frame 22 cooperate to form right panel 26, while central section 48 extends freely between sleeves 44, 46. Panels 24, 26 are shaped to effectively insert compression body 14 into a mother's pelvic cavity, and slide panels 24, 26 around an infant for swaddling or wrapping compression body 14 around an infant. (FIGS. 4-6).

Flexible sheet 18 is considered fully opened, such that compression body 14 and device 10 are fully deployed, when central section 48 has no portions overlapping itself and panels 24, 26. Compression body 14 includes an upper surface 50a and a lower surface 50b that oppose one another while compression body 14 is fully deployed such that one of surfaces 50a, 50b is facing an infant, and the other of surfaces 50a, 50b is contacting the infant's mother. (FIG. 4). Such surfaces 50a, 50b are defined by panels 24, 26 and central section 48 of flexible sheet 18.

Panels 24, 26 may each have a length L corresponding to about the length of a human's vaginal canal during labor. (FIG. 6). For example, the length L of panels 24, 26 may range from about 3 inches to about 6 inches, such as about 4.5 inches. Panels 24, 26 may each have a width that is less than or equal to a typical medical provider's index and middle finger together. (FIG. 17). For example, panels 24, 26 may each have a width that ranges from about 1 inch to about 3 inches, such as about 1.5 inches. The width and length of panels 24, 26 thus provide a sufficient surface area that can be effectively used to contact and compress an infant.

Left panel 24 defines a left edge portion 14a of compression body 14, and right panel 26 defines a right edge portion 14b of compression body 14. (FIG. 4). Lateral edge portions 14a, 14b face away from central section 48 of flexible sheet 18 while device 10 is fully deployed. Compression body 14 has an overall length extending between lateral edge portions 14a, 14b that is greater than an overall width W of flexible sheet 18, regardless of whether compression body 14 is under tension, such as when it is compressing an infant. (FIGS. 4, 6). The width W of flexible sheet 18 may be similar to the length L of panels 24, 26, and may correspond to about the length of a female's vaginal canal during labor. (FIG. 6). For example, the width W of flexible sheet 18 may range from about 2.75 inches to about 6.25 inches, such as about 4 inches. The overall length of compression body 14 corresponds to at least twice the typical obstetric conjugate of a human female. (FIG. 4). For example, an obstetric conjugate may typically be about 4.5 inches, and the overall length of compression body 14 may be about 11.25 inches.

Left manipulator arm 28 includes a left shaft 52 that is attached to left panel 24, and longitudinally extends in the same direction as left panel 24 to form a paddle shape in which left panel 24 is wider than left shaft 52. (FIG. 6). Specifically, left shaft 52 is straight and has a longitudinal axis 53a that is collinear with a longitudinal axis 53b of left panel 24. Right manipulator arm 30 similarly includes a right shaft 54 that is straight, attached to right panel 26, and has a longitudinal axis 55a that is collinear with a longitudinal axis 55b of right panel 26 to form a paddle shape in which right panel 26 is wider than right shaft 54. Shafts 52, 54 may each have a length that ranges from about 5 inches to about 12 inches, such as about 7 inches, that allows compression body 14 to be inserted into a human's vaginal canal to a depth that can treat shoulder dystocia.

Left manipulator arm 28 includes a left handle 56 and a left joint or linkage 58 that couples left handle 56 to a proximal portion 52a of left shaft 52. (FIG. 4). Likewise, right manipulator arm 30 includes a right handle 60 coupled to a proximal portion 54a of right shaft 54 via a right joint or linkage 62. Right handle 60 is adapted to be held by either a medical provider's left or right hand, and left handle 56 is adapted to be held by the provider's other hand.

Left handle 56 longitudinally extends in a direction laterally away from left shaft 52 and laterally beyond compression body's left edge portion 14a, and right handle 60 longitudinally extends in a direction laterally away from right shaft 54 and laterally beyond compression body's right edge portion 14b. (FIGS. 4, 6-8). Handles 56, 60 longitudinally extend away from respective shafts 52, 54 at about a 90 degree angle. As such, left handle 56 has a longitudinal axis that is perpendicular to left shaft's longitudinal axis 53a, and right handle 60 has a longitudinal axis that is perpendicular to right shaft's longitudinal axis 55a. (FIG. 6).

Handles 56, 60 may each have a length ranging from about 4 inches to about 7 inches, such as about 5 inches. (FIG. 4). Handles 56, 60 may be made of a solid surface material that is ergonomically shaped so that a medical provider can readily grasp them.

Left shaft 52 includes a distal portion 52b attached to a proximal portion 24a of left panel 24, which is defined by a portion of left frame 20. (FIG. 4). Left panel 24 includes opposing edge portions disposed in proximal portion 24a that taper toward longitudinal axes 53a, 53b as they approach left shaft 52. (FIG. 6). Left shaft 52 includes opposing edge portions that align with left panel's 24 opposing edge portions to form a smooth transition between left panel 24 and left manipulator arm 28. (FIGS. 4-6). Right shaft 54 similarly includes a distal portion 54b coupled to a proximal portion 26a of right panel 26, which is defined by a portion of right frame 22, and similarly includes opposing edge portions that align with respective opposing edge portions of right panel 26 to form a smooth transition between right panel 26 and right manipulator arm 30. The edge portions of right panel 26 that are disposed in proximal portion 26a also similarly taper toward longitudinal axes 55a, 55b as they approach right shaft 54. (FIG. 6).

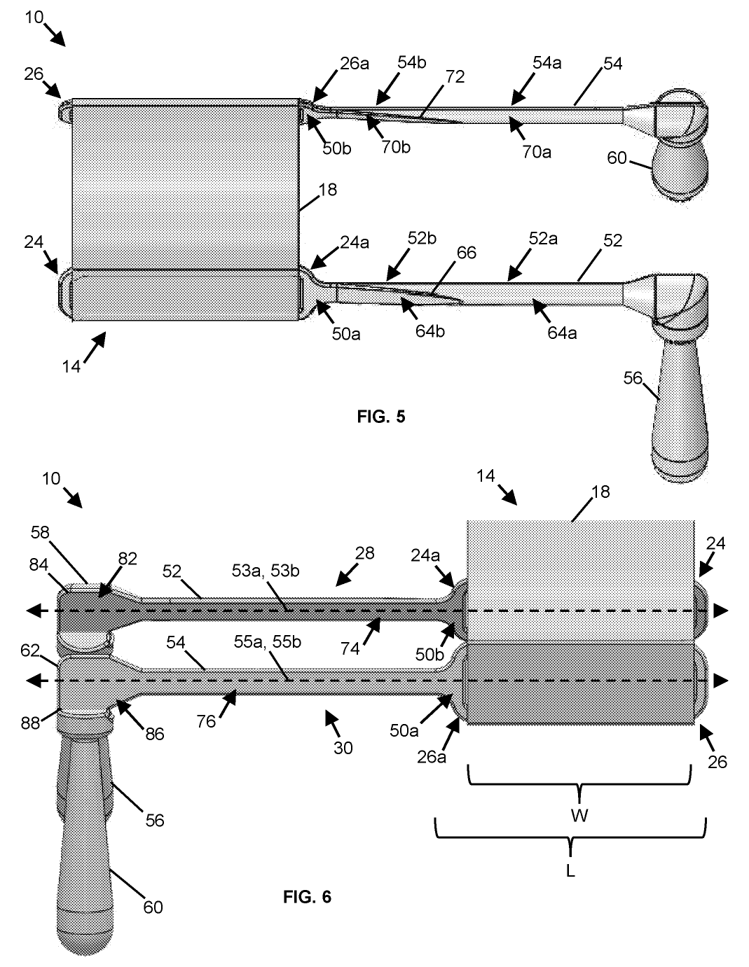

Referring to FIGS. 4 and 5, left shaft 52 defines an upper surface having an arcuate upper surface 64a and a sloped or ramped upper surface 64b. Sloped surface 64b extends in left shaft's distal portion 52b from a curved edge 66 dividing surfaces 64a, 64b and toward the portion of compression body's upper surface 50a that is defined by left panel 24. Left shaft's upper surface that is disposed in proximal portion 52a is entirely defined by arcuate surface 64a such that proximal portion 52a has a half-cylinder shape.

Right shaft 54 defines a lower surface that is similar to left shaft's upper surface in that it includes an arcuate lower surface 70a and a sloped or ramped lower surface 70b. (FIGS. 4, 5). Right sloped surface 70b extends from a curved edge 72 dividing surfaces 70a, 70b and toward the portion of compression body's lower surface 50b that is defined by right panel 26. Right shaft's 54 lower surface that is disposed in proximal portion 54a is entirely defined by arcuate surface 70a such that proximal portion 54a has a half-cylinder shape.

Sloped surfaces 64b, 70b are flat and may be angled between 90 and 180 degrees, such as about 105 degrees, relative to respective surfaces 50a, 50b. (FIGS. 4, 5). As such, the thickness of shafts' distal portions 52b, 54b may gradually increase as they approach respective curved edges 66, 72. For example, panels' proximal portions 24a, 26a may each have a thickness of about 0.10 inch, and shafts' proximal portions 52a, 54a may each have a thickness of about 0.25 inch. Therefore, sloped surfaces 64b, 70b may be tapered such that shafts' distal portions 52b, 54b each gradually increases in thickness from about 0.10 inch to about 0.25 inch.

Referring to FIG. 6, left shaft 52 defines a lower surface 74 that is planar and aligns with the portion of compression body's lower surface 50b defined by left panel 24. Right shaft 54 defines an upper surface 76 that is similar to left shaft's lower surface 74 in that it is planar and aligns with the portion of compression body's upper surface 50a defined by right panel 26.

As shown in FIGS. 4-6, left shaft's surfaces 64a, 64b, 74 align with respective surfaces 50a, 50b of compression body 14 to form a smooth transition between left panel 24 and left manipulator arm 28. Likewise, right shaft's surfaces 70a, 70b, 76 align with respective surfaces 50a, 50b to form a smooth transition between right panel 26 and right manipulator arm 30.

Manipulator arms 28, 30 and panels 24, 26 may have atraumatic shapes to facilitate injury prevention of the mother and infant. (FIGS. 4-6). For example, the tapering of panels 24, 26 at panels' proximal portions 24a, 26a, the rounded distal edge portions of panels 24, 26, and the transitions between surfaces 50a, 50b, 64a, 64b, 70a, 70b, 74, 76 provide atraumatic shapes that may prevent maternal and/or neonatal tissue damage.

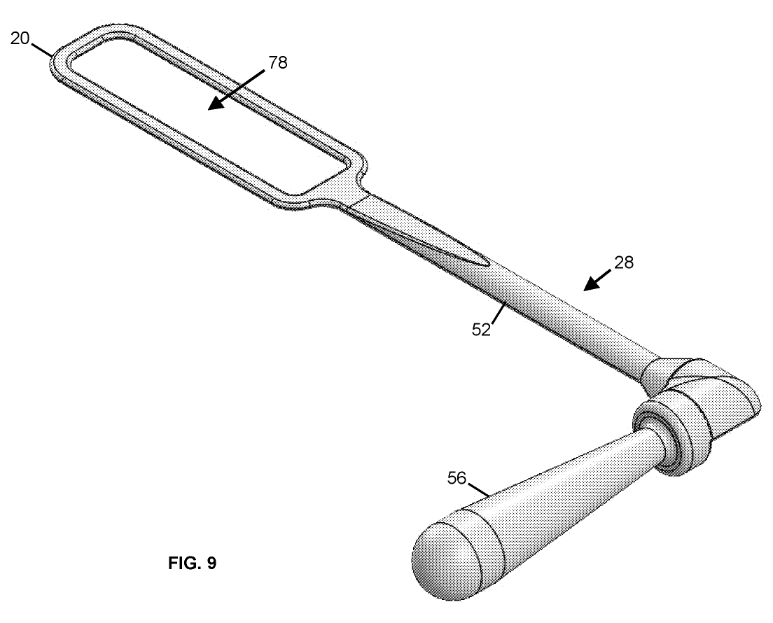

Referring to FIG. 9, left frame 20 and left manipulator arm 28 are shown without flexible sheet 18 and without right frame 22 and right manipulator arm 30. Left frame 20 defines an elongated through hole 78 such that left frame 20 has an elongated ring shape. Right frame 22 and right manipulator arm 30 are identical to left frame 20 and left manipulator arm 28, respectively.

Left sleeve 44 wraps around through hole 78 such that through hole 78 is covered, as shown in FIGS. 4-6. Panel 24 thus has a resilient portion in which left sleeve 44 is extending over through hole 78 and a supported portion in which left sleeve 44 is extending over frame 20. For example, while lower surface 50b of compression body 14 is being used to contact and compress an infant, the portion of lower surface 50b extending over frame 20 is more rigid than the portion of lower surface 50b extending over through hole 78 to compress an infant's impacted body segment. The portion of lower surface 50b extending over through hole 78 also aids in compressing the infant by providing additional surface area that is deformable, which may mitigate injury of the infant's impacted body segment. For example, this can aid in positioning panel 24 around a shoulder of the infant. Right sleeve 46 similarly wraps around right frame's 22 through hole (not shown) such that right panel 26 has a similar function as left panel 24.

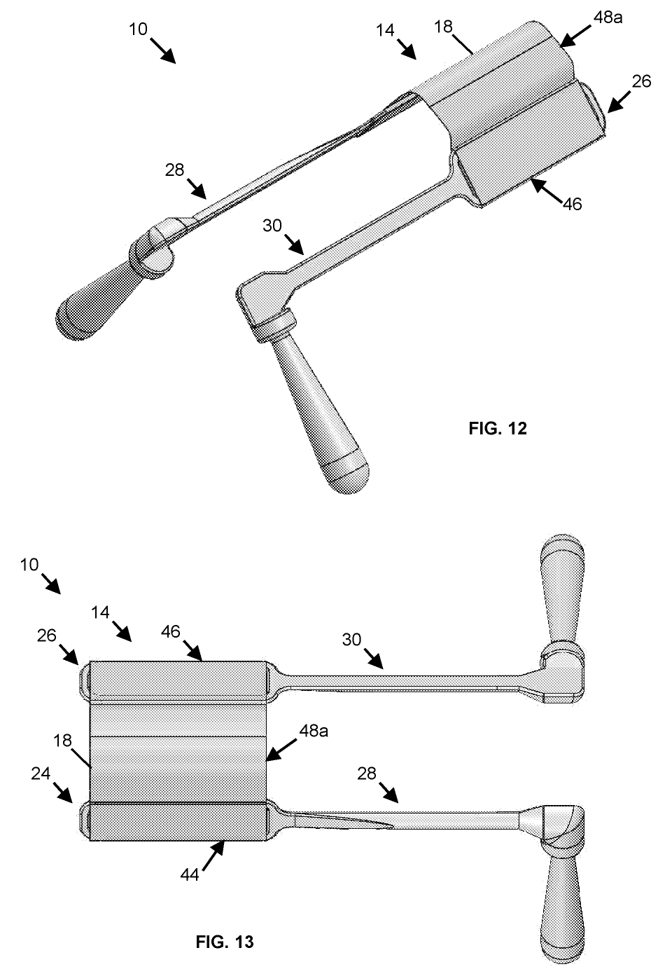

Referring to FIGS. 12-15, device 10 is shown in a partially deployed configuration in which flexible sheet 18 is partially opened and not yet usable to compress an infant. Device 10 may be in this position after compression body 14 has been inserted into a mother's pelvic cavity, and after manipulator arms 28, 30 have been moved to deploy a portion 48a of flexible sheet's central section 48. Deployed portion 48a corresponds to the portion of central section 48 that does not overlap itself and panels 24, 26.

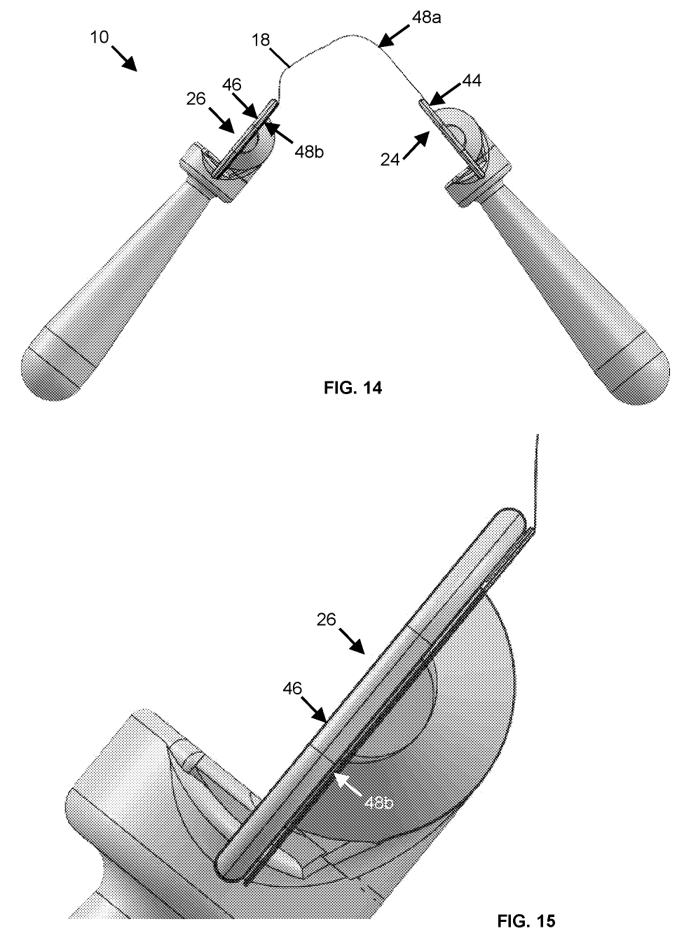

As best shown in FIGS. 14 and 15, central section 48 includes an undeployed or closed portion 48b that overlaps right panel 26. To position device 10 in the deployed configuration shown in FIGS. 4-8 such that it is usable to compress an infant, manipulator arms 28, 30 need to be further moved to cause closed portion 48b to move laterally away from right panel 26 and no longer overlap with panel 26.

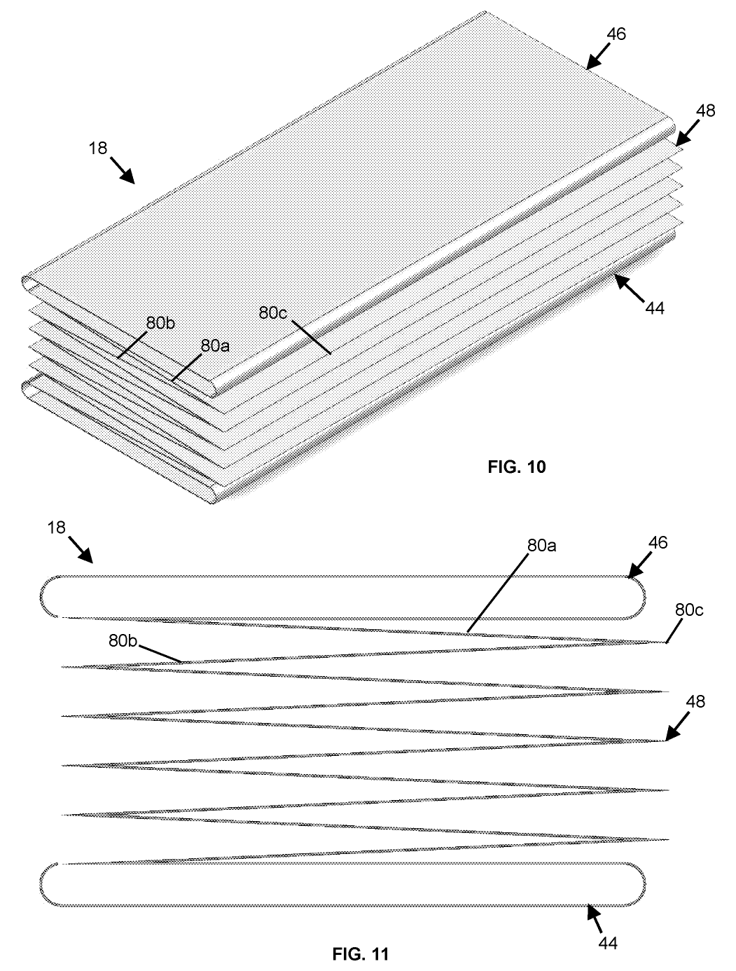

Referring to FIGS. 10 and 11, flexible sheet 18 is shown in a fully undeployed or closed configuration in which the entirety of central section 48 is overlapping itself in an accordion or zig-zag shape. Thus, flexible sheet 18 in that configuration is not usable to compress an infant. The accordion shape of central section 48 is defined by multiple segments that are divided by folds. For example, central section 48 includes a first segment 80a coupled to right sleeve 46, and a second segment 80b coupled to first segment 80a and divided from first segment 80a by fold 80c. Closed portion 48b of central section 48 shown in FIGS. 14 and 15 may correspond to first and second segments 80a, 80b in the undeployed configuration as shown in FIGS. 10 and 11.

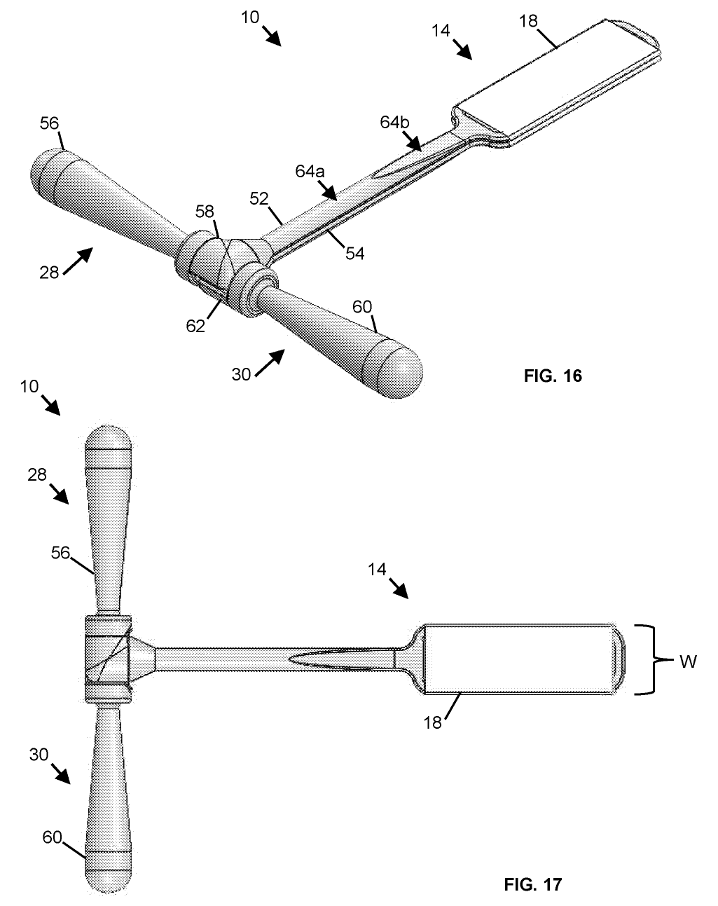

Referring to FIGS. 16-19, device 10 is shown in an undeployed or closed configuration in which flexible sheet 18 is fully closed and not yet usable to compress an infant. For example, flexible sheet 18 may be positioned in the accordion-style configuration shown in FIGS. 10 and 11. Device 10 may be in the undeployed configuration when compression body 14 is in the process of being inserted into a mother's pelvic cavity. Once compression body 14 is properly positioned in the pelvic cavity, a medical provider can move manipulator arms 28, 30 to place device 10 in a fully deployed configuration, such as shown in FIGS. 4-8. This process is discussed in greater detail below in connection with FIGS. 20-23.

Flexible sheet 18 folded in an accordion-style or zig-zag manner while in the undeployed configuration allows device 10 to be rapidly deployed. (FIGS. 16-19). For example, flexible sheet 18 folded in an accordion-style manner can readily be separated to wrap around an infant by moving manipulator arms 28, 30.

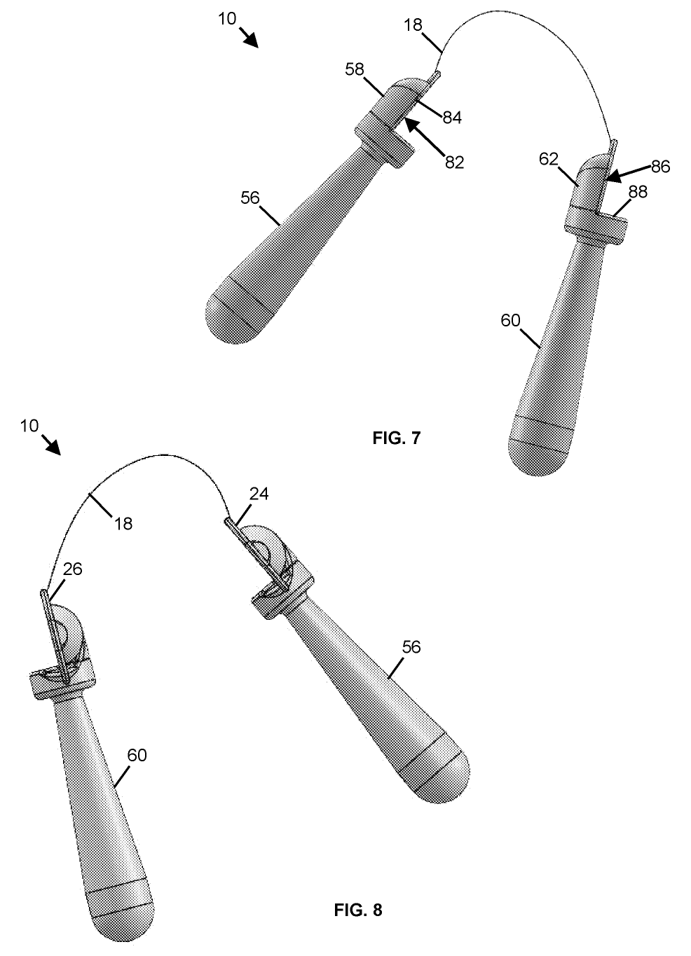

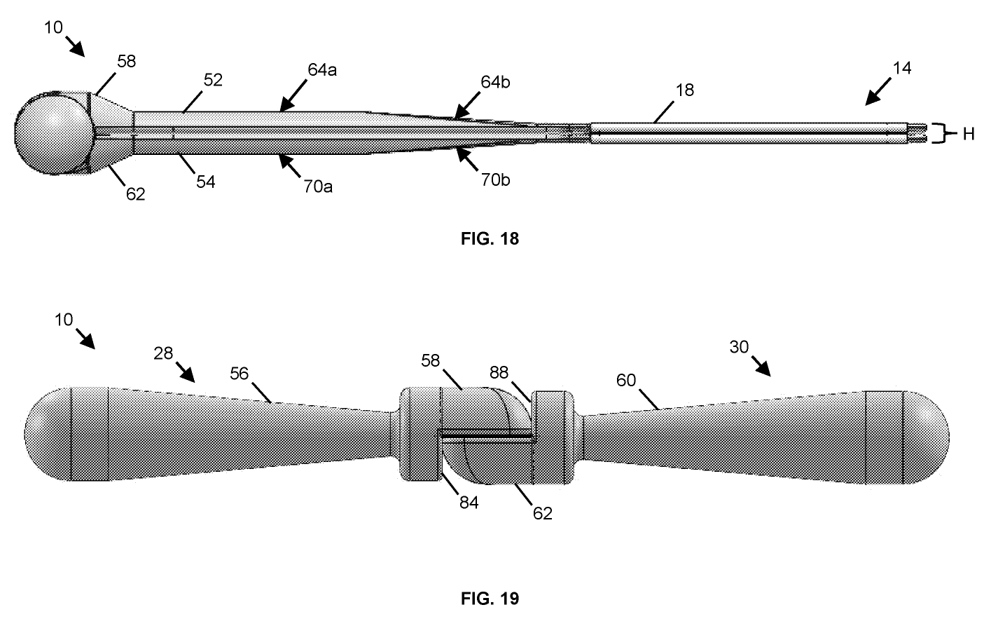

Device 10 in the undeployed configuration has panels 24, 26 overlapping one another, and manipulator arms 28, 30 engaged with one another. (FIGS. 16-19). In particular, left shaft's lower planar surface 74 engages with right shaft's upper planar surface 76. (FIGS. 6, 16-19). Joints 58, 62 of manipulator arms 28, 30 are also engaged with one another while device 10 is in the undeployed configuration. (FIGS. 16-19). As best shown in FIGS. 6 and 7, left joint 58 includes a lower surface 82 that defines a notch 84 facing away from left handle 56, and right joint 62 includes an upper surface 86 that defines a notch 88 facing away from right handle 60. Notches 84, 88 engage with one another while device 10 is in the undeployed configuration. (FIGS. 16, 19). The surfaces of joints 58, 62 opposing respective notches 84, 88 may form a conical shape. The stacked or undeployed configuration of compression body 14 has a height H sized to be inserted into a human's vaginal canal, such as less than or equal to a thickness of a typical human surgeon’s finger. (FIG. 18). For example, the height may range from about 0.4 inch to about 1.5 inches, such as about 0.75 inch.

When device 10 is in the undeployed configuration such that panels 24, 26 are stacked, the width W of compression body 14 in the stacked configuration may correspond to the width of panels 24, 26, such as about 1.5 inches. (FIG. 17). This allows compression body 14 to be readily inserted into a vaginal canal during difficult births, such as shoulder dystocia scenarios.

Device 10 has an overall length extending from a proximal end of left joint 58 or right joint 62 to a distal end of left panel 24 or right panel 26, respectively. The overall length may range from about 10 inches to about 13 inches, such as about 11 inches.

Referring to FIGS. 20-23, device 10 is shown in different positions throughout the process of it being used to treat shoulder dystocia of infant 12 in a pelvic cavity. Device 10 and infant 12 are shown relative to pelvis 16a and a vaginal canal 16b. The process of using device 10 to treat shoulder dystocia of infant 12 begins by inserting compression body 14 into the pelvic cavity via vaginal canal 16b. (FIGS. 20, 21). Device 10 is inserted while in an undeployed configuration in which flexible sheet 18 is fully closed. (FIGS. 16-21).

As shown in FIGS. 20-23, infant 12 is in a left occiput transverse (LOT) position, and undeployed compression body 14 is adapted to be guided into the anatomical left anterior portion of the mother’s pelvic cavity while in that LOT position. For example, a medical provider may use their right hand to insert undeployed compression body 14 with their index and middle finger, supported by their thumb. The operator may continue to insert undeployed device 10 through vaginal canal 16b until undeployed compression body 14 is engaged with the infant’s back in the anterior portion of pelvis 16a, as shown in FIGS. 20 and 21. Compression body 14 is inserted such that it does not engage with the mother's anterior arch and pubic symphysis. Birthing device 10 is adapted to be inserted into the mother's pelvic cavity at a depth that does not exceed the depth of vaginal canal 16b, such as about 4.5 inches. Alternatively or additionally, device 10 may be adapted to not be inserted beyond panels 24, 26 or beyond a midpoint of shaft's distal portions 52b, 54b. For example, device 10 is shown inserted not beyond panels 24, 26 in FIGS. 20 and 21.

After device 10 is engaged with the infant’s back, the medical provider can begin deploying device 10 by separating manipulator arms 28, 30. (FIG. 22). For example, the provider's left hand may hold left handle 56, and the provider's right hand may hold right handle 60. The provider's left hand may move left handle 56 to position left panel 24 between the upper body of infant 12, such as a bony prominence of the humerus of infant 12, and the anterior portion of pelvis 16a, such as the mother's pubic symphysis. The provider's right hand may move right handle 60 to position right panel 26 between the upper body of infant 12, such as a bony prominence of the humerus of infant 12, and the posterior portion of pelvis 16a, such as the mother's perineum.

Panels 24, 26 are further positioned such that left panel 24 engages with an anterior portion of the upper body of infant 12, such as a bony prominence of the anterior humerus of infant 12, and right panel 26 engages with a posterior portion of the upper body of infant 12, such as a bony prominence of the posterior humerus of infant 12. In some examples, flexible sheet 18 may not be fully opened or deployed when panels 24, 26 are engaged with the bony prominences of respective anterior and posterior humerus of infant 12. In that case, the medical provider may move manipulator arms 28, 30 away from one another to fully open flexible sheet 18, and re-engage with the bony prominences of respective anterior and posterior humerus of infant 12 to fully deploy device 10.

FIG. 22 shows device 10 fully deployed and engaged with the bony prominences of respective anterior and posterior humerus of infant 12. The medical provider may then apply gentle compressive force by moving handles 56, 60 toward the midline of infant 12 to tighten and engage compression body 14 around the upper body of infant 12, such as a shoulder girdle of infant 12. This application of gentle compressive force aids the medical provider in collapsing the shoulder girdle impacted by the mother's pubic symphysis. FIG. 23 shows device 10 while compressive force is being applied in which handles 56, 60 are overlapping one another.

After the shoulder girdle of infant 12 has been collapsed, the medical provider may apply a rotational force, in addition to the compressive force, to remove the shoulder of infant 12 from the pubic symphysis and present the infant in a face down (cephalic) position, as shown in FIG. 23. This may be accomplished by rotating handles 56, 60 counterclockwise. Once the shoulder of infant 12 has been removed from the mother's pubic symphysis, the medical provider may apply an axial force, in addition to the compressive force, on the infant 12 by adduction of handles 56, 60 for delivery of infant 12 and resolution of shoulder dystocia.

Manipulator arms 28, 30 and frames 20, 22 are made of a material more rigid than flexible sheet 18. However, arms 28, 30 and frames 20, 22 may have some level of flexibility, such as for allowing compression body 14 to effectively maneuver after its insertion to treat shoulder dystocia. For example, arms 28, 30 and frames 20, 22 may be made of or include an atraumatic and biocompatible polymer, such as polylactic acid (PLA). Alternatively or additionally, arms 28, 30 and frames 20, 22 may be made of or include metal (e.g., stainless steel or titanium), polyether ether ketone (PEEK), high-density polyethylene (HDPE), polytetrafluoroethylene (PTFE), nylon, polyamide, polyether block amide, polyvinyl chloride (PVC), thermoplastic polyurethane, silicone, and/or another high strength, flexible plastic.

Frames 20, 22 of compression body 14 may include a coating that is adapted to prevent maternal and/or neonatal tissue damage. For example, frames 20, 22 may be coated in an atraumatic soft polymer, such as silicone. Alternatively or additionally, frames 20, 22 may be coated in a foam, plastic, and/or biocompatible lubrication coating.

Panels 24, 26 may each have a thickness ranging from about 0.08 inch to about 0.15 inch, such as about 0.10 inch. Shafts 52, 54 may each have a thickness ranging from about 0.10 inch to about 0.30 inch, such as about 0.25 inch at proximal portions 52a, 54a.

Flexible sheet 18 is made of a material that is more flexible than manipulator arms 28, 30 and frames 20, 22. The material of sheet 18 provides a high tensile strength and high flexibility such that sheet 18 can conform to both neonatal and maternal anatomy. For example, sheet 18 may be made of or include a soft polymer that is atraumatic and biocompatible, such as a low density polyethylene (LDPE). Alternatively or additionally, sheet 18 may be made of or include any other suitable material, such as elastic polytetrafluoroethylene (ePTFE) and/or silicone. Flexible sheet 18 has a thickness ranging from about 0.5 millimeter to about 4.0 millimeters, such as about 0.8 millimeter.

Sleeves 44, 46 of flexible sheet 18 may be attached to respective frames 20, 22 by some form of manufacture that prevents them slipping off distal ends of frames 20, 22, such as using ultrasonic welding and/or biocompatible adhesives.

Device 10 may be adapted for single-use applications, and device 10 may be made of or include suitable single-use materials. Alternatively, at least a portion of device 10 may be reusable. For example, manipulator arms 28, 30 and frames 20, 22 may be reusable, and made of or include suitable reusable materials that allow for sterilization, whereas flexible sheet 18 may adapted for single-use applications and selectively attached to frames 20, 22.

Accordingly, the birthing device has a compression body that swaddles and compresses an infant in a mother's pelvic cavity to resolve labor complications, such as shoulder dystocia. The compression body has panels providing sufficient surface areas that can be effectively used to contact and compress the infant. The compression body also has a flexible sheet to facilitate compression of the infant, which may define portions of the panels. The panels may provide an atraumatic shape that allows panels to be readily slid between the infant and mother's vaginal canal to wrap the compression body around the infant.

Although FIGS. 1-3 and FIGS. 20-23 are showing infants 12, 33 in a left occiput transverse (LOT) position, it should be understood devices 10, 32 can be used to treat shoulder dystocia of infants in other positions, such as a right occiput transverse (ROT) position. Positioning and movement of manipulator arms 28, 30 and panels 24, 26 may be adjusted for infants in different positions, which will be understood by one of ordinary skill in the art.

Although examples of devices 10, 32 are shown in the figures, one or more of the illustrated elements may be combined, divided, re-arranged, omitted, and/or implemented in any other way. For example, frames 20, 22 and manipulator arms 28, 30 may be nonidentical. In another example, through hole 78 of left frame 20 and the through hole (not shown) of right frame 22 may be omitted, and frames 20, 22 may define the entirety of respective panels 24, 26. In that example, flexible sheet 18 may omit sleeves 44, 46, and central section 48 may be attached to frames 20, 22. Although sleeves 44, 46 and central section 48 form a single, unitary flexible sheet 18, it will be appreciated that left sleeve 44, right sleeve 46, and/or section 48 may be modified such that they are separate from one another and independently coupled to frames 20, 22.

It will be appreciated that one or more elements of devices 10, 32 may be modified to have different sizes or dimensions, such as for use with non-human mammals. For example, device 10 may be modified for use with assisting a difficult birth of a particular non-human mammal, and device 10 may be sized to accommodate that non-human mammal's anatomy.

Although handles 56, 60 are shown perpendicular to respective shafts' longitudinal axes 53a, 55a, handles 56, 60 may be oriented in any other suitable way. For example, left handle 56 may be modified such that there is about a 70 degree angle between longitudinal axis of left handle 56 and left shaft's longitudinal axis 53a. As a result, there is an acute angle between left handle 56 and an outer edge portion of left shaft 52. In another example, left handle 56 may be modified such that left handle 56 has a longitudinal axis collinear with axes 53a, 53b of left shaft 52 and left panel 24. Thus, device 10 may be modified to have an angle between the longitudinal axis of left handle 56 and longitudinal axis 53a of left shaft 52 that ranges from about 70 degrees to about 180 degrees. The orientation of right handle 60 can also be modified relative to right shaft 54 in like manners.

In another example of modifying device 10, although panels 24, 26 are shown having longitudinal axes 53b, 55b collinear with shafts' axes 53a, 55a, axes 53b, 55b of panels 24, 26 may be oriented in any other suitable way. Left panel 24 may be modified such that there is about a 120 degree angle between left panel's longitudinal axis 53b and left shaft's longitudinal axis 53a, assuming collinear longitudinal axes 53a, 53b have a 180 degree angle. As a result, left panel's longitudinal axis 53a is directed inward. In another example, left panel 24 may be modified such that there is about a 190 degree angle between left panel's longitudinal axis 53b and left shaft's longitudinal axis 53a, assuming collinear longitudinal axes 53a, 53b have a 180 degree angle. As a result, left panel's longitudinal axis 53b is directed outward. Thus, device 10 may be modified to have an angle between longitudinal axis 53a of left shaft 52 and longitudinal axis 53b of left panel 24 that ranges from about 120 degrees to about 190 degrees. The orientation of right panel 26 can also be modified relative to right shaft 54 in like manners.

It is noted that devices 10, 32 shown in the figures may include one or more elements in addition to and/or instead of the illustrated elements, and/or may include more than one of the illustrated elements. For instance, it should also be appreciated that devices 10, 32 could be used by non-organic operators, such as a surgical robot, and in some examples, devices 10, 32 may modified for use with such non-organic operators. For example, handles 56, 60 may be replaced with an instrument interface suitable for operation by a particular non-organic operator, such as by implementing mechanical, electrical, and/or telemetry technologies.

Devices 10, 32 may also be modified to include one or more sensors that provide feedback to an operator. For example, device 10 may include one or more pressure sensors, temperature sensors, accelerometers, and/or image sensors (e.g., cameras).

Connection relationships between elements are described herein using various terms such as, for example, "coupled," "disposed," "attached," "engaged," and the like. As used herein, connection relationships can be direct relationships and/or indirect relationships where one or more intervening elements are between the first and second elements.

Spatial relationships of elements are described herein using various terms such as, for example, "lateral," "upper," "lower," "left," "right," "front," "back," "opposing," "outward," "inward," and the like. As used herein, spatial relationships of the elements do not limit the orientations of the elements as other orientations of the elements may be used.

It should be understood that "including", "comprising", and "having" (and all other forms, such as tenses) are used herein to be open-ended terms. Thus, whenever a claim recites any form of "include", "comprise", or "have" (e.g., comprises, includes, has, comprising, including, having) as a preamble or within a claim recitation of any kind, it is to be understood that additional elements, terms, etc. may be present without falling outside the scope of the corresponding claim.

As used herein, singular references (e.g., "a'', "an'', ''first'', "second") do not exclude a plurality. The term "a" or "an" entity refers to one or more of that entity. The terms "a" (or "an"), "one or more", and "at least one" can be used interchangeably. The term "and/or" when used in a form such as, for example, A, B, and/or C refers to any combination or subset of A, B, C such as (1) A alone, (2) B alone, (3) C alone, (4) A with B, (5) A with C, (6) B with C, and (7) A with B and with C.

Changes and modifications in the specifically described examples can be carried out without departing from the principles of the present disclosure which is intended to be limited only by the scope of the appended claims, as interpreted according to the principles of patent law including the doctrine of equivalents.

1. A device for treating shoulder dystocia, the device comprising:
a pair of arms;
a pair of panels coupled to respective ones of said arms; and
a flexible sheet coupled to said panels;
wherein said panels and said flexible sheet are adapted to cooperate with one another to manipulate an infant in a pelvic cavity.

2. The device of claim 1, wherein:
said flexible sheet is a third flexible sheet;
a first of said panels comprises a first frame coupled to a first of said arms, and a first flexible sheet coupled to said first frame; and
a second of said panels comprises a second frame coupled to a second of said arms, and a second flexible sheet coupled to said second frame.

3. The device of claim 2, wherein said first frame defines a first through hole, and said first flexible sheet covers at least a portion of said first through hole, wherein said second frame defines a second through hole, and said second flexible sheet covers at least a portion of said second through hole.

4. The device of claim 3, wherein said first and second through holes have an elongated shape.

5. The device of claim 4, wherein said first and second frames are planar and said first, second, and third flexible sheets are rectangular shaped.

6. The device of claim 2, wherein said first, second, and third flexible sheets are unitarily formed.

7. The device of claim 1, wherein said panels include respective lateral edge portions, and said device has a first width extending between said lateral edge portions, wherein said flexible sheet has a second width extending along a first of said lateral edge portions, and said first width is greater than said second width.

8. The device of claim 1, wherein said panels and said flexible sheet are sized to cooperate with one another to compress a human infant in a human's pelvic cavity.

9. The device of claim 1, wherein a first of said arms comprises a first straight shaft coupled to a first of said panels, and a second of said arms comprises a second straight shaft coupled to a second of said panels, wherein said first straight shaft and said first panel have respective longitudinal axes that are collinear, and said second straight shaft and said second panel have respective longitudinal axes that are collinear.

10. The device of claim 1, wherein a first of said arms comprises a first shaft coupled to a first of said panels, and a second of said arms comprises a second shaft coupled to a second of said panels, wherein said first and second arms comprise respective first and second handles coupled to respective ones of said first and second shafts, wherein said first handle longitudinally extends in a first direction laterally away from said first shaft, and said second handle longitudinally extends in a second direction laterally away from said second shaft.

11. The device of claim 1, wherein a first of said arms comprises a first shaft defining opposing first and second surfaces, wherein said first surface includes an arcuate surface, and said second surface is planar, wherein a second of said arms comprises a second shaft defining opposing third and fourth surfaces, wherein said third surface includes an arcuate surface, and said fourth surface is planar.

12. The device of claim 11, wherein said first and second shafts are straight.

13. The device of claim 1, wherein said arms define respective notches adapted to engage with one another.

14. The device of claim 13, wherein a first of said arms comprises a first straight shaft coupled to a first of said panels, a first joint defining a first of said notches, and a first handle coupled to said first straight shaft via said first joint, wherein a second of said arms comprises a second straight shaft coupled to a second of said panels, a second joint defining a second of said notches, and a second handle coupled to said second straight shaft via said second joint.

15. The device of claim 1, wherein said flexible sheet defines a plurality of segments that are adapted to be stacked in an accordion shape.

16. A device for treating shoulder dystocia, the device comprising:
a first panel including a first planar frame defining a first through hole, and a first flexible sheet coupled to said first planar frame and covering said first through hole;
a first arm including a first straight shaft coupled to said first planar frame, a first joint defining a first notch, and a first handle coupled to said first straight shaft via said first joint;
a second panel including a second planar frame defining a second through hole, and a second flexible sheet coupled to said second planar frame and covering said second through hole;
a second arm including a second straight shaft coupled to said second planar frame, a second joint defining a second notch adapted to engage with said first notch, and a second handle coupled to said second straight shaft via said second joint; and
a third flexible sheet unitarily formed with said first flexible sheet and said second flexible sheet, and said third flexible sheet defining a plurality of segments that are adapted to be stacked in an accordion shape;
wherein said first straight shaft and said first planar frame have respective longitudinal axes that are collinear, and said second straight shaft and said second planar frame have respective longitudinal axes that are collinear;
wherein said first panel, said second panel, and said third flexible sheet are adapted to cooperate with one another to manipulate an infant in a pelvic cavity.
